(19) United States
(12) Patent Application Publication
(10) Pub. No.: US 2010/0286636 A1
(43) Pub. Date: Nov. 11, 2010

(54) DISPOSABLE NOZZLE

(75) Inventor: MARKUS BRAENDLI, Erlinsbach (CH)

Correspondence Address: DORITY & MANNING, P.A., POST OFFICE BOX 1449, GREENVILLE, SC, 29602-1449, US

(73) Assignee: MEDAXIS AG, Aarau (CH)

(21) Appl. No.: 12/463,483

(22) Filed: May 11, 2009

(51) Int. Cl. A61M 35/00

(52) U.S. Cl. 604/310

(57) ABSTRACT

The present invention relates to a disposable nozzle for inserting into a hand piece for treating and cleaning wounds by means of a highly focused high-pressure micro water jet. The disposable nozzle comprises a cylindrical nozzle body having a centrally traversing orifice body duct, which is laterally closed, as well as a cylindrical orifice with a centrally traversing orifice duct. The orifice is disposed in a recess that is centrally arranged in the frontal terminal region of the nozzle body at the front side in such a manner that both ducts adjoin to each other in a coaxial manner. An O-ring is disposed in the recess between the nozzle body and the orifice in a clamping manner, wherein the O-ring centrally retains the orifice in the nozzle body.

### CROSS-REFERENCE TO RELATED APPLICATIONS

N/A

### STATEMENT REGARDING FEDERALLY SPONSORED RESEARCH OR DEVELOPMENT

N/A

### BACKGROUND OF THE INVENTION

The present invention relates to a disposable nozzle for the insertion into a hand piece for treating and cleaning wounds by a highly focused high-pressure micro water jet.

In a treatment, said water jet functions in place of a scalpel or a bone curette for the removal of tissues or secretions. To this end, a liquid pump applies a high hydraulic pressure onto a sterile surgical liquid, e.g., brine. The liquid flows through a reusable high-pressure lead with a hand piece, where the micro water jet is emitted through a sterile disposable nozzle.

In medical technology, such disposable nozzles are mainly used for the removal of fibrin coverings and necroses on chronic wounds. Before a treatment, such disposable nozzles are inserted into sterilized hand pieces designated for this purpose by the treating personal, and in the course of the application they create a highly focused high-pressure micro water jet with approx. 150 bars.

The micro water jet must flow very coherently over a wide range in order to achieve the necessary action. For this purpose, the disposable nozzles have an orifice with an orifice duct of approx. 0.009 mm in diameter at the front of their nozzle body proximal to the exit of the water jet. Usually, the orifices are cemented into the nozzle bodies. Conventional nozzle bodies are made from metal and are relatively expensive.

Following a treatment, all instruments are highly infected and must be cleaned and sterilized. However, due to its narrow duct it is not possible to clean the disposable nozzle in a sufficient manner, and therefore it must be discarded.

Such disposable nozzles are often adversely affected by drawbacks. On the one hand, they often clog since they are bombarded by contaminations splattering around in the course of a treatment. On the other hand, the liquid also often flows laterally past the nozzle body or laterally past the orifice and exits laterally. This results in a micro water jet which is inferior to useless.

FIG. 1 shows a schematic representation in a sectional view of a disposable nozzle 1 for inserting into a hand piece 17 for treating and cleaning wounds by a highly focused high-pressure micro water jet according to the prior art. Said disposable nozzle 1 comprises a cylindrical nozzle body 2 having a centrally traversing nozzle body duct 3 which is laterally closed. The disposable nozzle 1 has a front terminal region 12, which is proximal to the exit of the water jet, and a rear terminal region 13 that is distal to the exit of the water jet. In the front terminal region 12 of the nozzle body 2, a recess 6 is disposed in the center at the front side. A cylindrical orifice 4 with a centrally traversing orifice duct 5 is arranged in the recess 6. Orifice 4 is attached in recess 6 in such a manner that duct 3 and duct 5 become adjoined to each other in essentially a coaxial manner.

Generally, the orifice 4 is attached at the nozzle body 2 by means of an adhesive. However, if the adhesive sticks only insufficiently, the high pressure may eject the orifice 4 from the nozzle body 2 into the wound. Moreover, it has been proven that when relying on adhesive to attach orifice 4 to the nozzle body 2, no adequate centering between nozzle body 2 and nozzle orifice 4 can reliably be achieved. The resulting inadequate centering results in a high rate of deficient disposable nozzles 1 produced in this manner. Additionally, it is often the case that liquid which is laterally exiting outside of orifice 4 due to a leaky adhesive exerts an adhesion action onto the micro water jet 16 which results in an inoperative stream of micro water jet 16.

In the rear terminal region 13 of the nozzle body 2, an 0-ring 10 is disposed at the cylinder wall in order to prevent an invasion of bacteria into the hand piece. A large conical aperture 14 of the nozzle body duct 3 in the rear terminal region 13 of the nozzle body leads to a small remaining rear surface 15 of the nozzle body.

FIG. 2 shows the same nozzle body 2 as it is represented in FIG. 1. However, in FIG. 2 it is mounted in a hand piece 17 with a hand piece cap 19. Hand piece 17 and hand piece cap 19 may be screwed to each other and opened again by means of a thread 21 to insert the disposable nozzle 1 and to clean the components. A hand piece duct 18 enables the influx of liquid to the nozzle body duct 3. The hand piece cap 19 exhibits an aperture region 20 where the high-pressure micro water jet 16 exits.

The disposable nozzle 1 rests at its front terminal region 12 proximal to the exit of the water jet on a front stopper 8 and is supported at one shoulder on hand piece cap 19. At its rear terminal region 12 at the rear surface of the nozzle body 15, disposable nozzle 1 rests on a rear stopper 11 at the hand piece 17. Thus, the disposable nozzle 1 is fixedly mounted in hand piece 17 with cap 19.

This assembly situation has the disadvantage of a small guidance due to a high degree of clearance between the components. A partly sloped position of the disposable nozzle 1 in hand piece 17 cannot be avoided and also not be controlled. However, studies yielded that a highly precise centering of the orifice 4 in nozzle body 2 is essential for a high quality of micro water jet 16.

It has been proven that bacteria may reach into the rear region of hand piece 17 in spite of the rear O-ring 10. This poses a danger because bacteria may penetrate through the hand piece duct 18 into the liquid tank and may infect other patients at a later point of time.

Further, the orifice duct 5 quickly clogs. This clogging results partly from the fact that orifice 4 is bombarded by bouncing particles. This clogging of duct 5 may cause a build up of high pressure that may eject the orifice 4 from the nozzle body 2 into the wound if the adhesive sticks only insufficiently. Finally, this and other reasons add to the fact that micro water jet 16 often does not fulfill the requirements.

It has to be noted, that a replacement of disposable nozzle 1 always means a longer treatment period for the patient and finally results in a loss of trust into the treatment method used. Additionally, the higher personnel and treatment costs of the longer treatment period and the costs for the disposable nozzles to be replaced have to be paid.

### BRIEF OBJECTS OF THE INVENTION

A principal object of the present invention is to provide for the insertion into a hand piece for treating and cleaning wounds by a highly focused high-pressure micro water jet, a disposable nozzle that provides a higher safety and reliability with respect to the quality of the micro water jet.

Another principal object of the present invention is to provide for the insertion into a hand piece for treating and cleaning wounds by a highly focused high-pressure micro water jet, a disposable nozzle that can be provided at reduced cost.

A further principal object of the present invention is to provide for the insertion into a hand piece for treating and cleaning wounds by a highly focused high-pressure micro water jet, a disposable nozzle comprising a cylindrical nozzle body having a centrally traversing nozzle body duct, which is laterally closed, and a cylindrical orifice having a centrally traversing orifice duct, wherein the orifice is disposed in a recess centrally arranged in the front terminal region of the nozzle body at the front side in such a manner, that both ducts adjoin to each other in a coaxial manner.

An additional principal object of the present invention is to provide a hand piece for treating and cleaning wounds by a highly focused high-pressure micro water jet, wherein the hand piece includes a disposable nozzle in accordance with the specification described herein.

The objects are solved by the disclosure described herein.

The idea underlying the present invention resides in the fact that the different ducts, through which the liquid flows to its exit, have a highly precise arrangement with respect to each other in the center. According to the present invention, the orifice is attached with an O-ring in a self-centering and sufficiently clamping manner in a recess in the nozzle body designated for this purpose in order to ensure such a centering. Thus, it is unnecessary to use an adhesive between nozzle body and orifice, and the O-ring protrudes to provide a better seal against the hand piece.

Presently preferred embodiments are mentioned in the description below and include for example the additional centering of the nozzle body in the hand piece by two O-rings, one of which being in the front and the other in the rear region of the nozzle body, respectively.

Additional objects and advantages of the invention will be set forth in part in the description that follows, and in part will be obvious from the description, or may be learned by practice of the invention. The objects and advantages of the invention may be realized and attained by means of the instrumentalities and combinations particularly pointed out in the appended claims.

### BRIEF DESCRIPTION OF THE DRAWINGS

In the following, the invention is explained in more detail with respect to the exemplary embodiments depicted in the drawings. In all figures, the same reference numerals are always used for the same components.

In the drawings, there is:

FIG. 1 depicts a schematic representation in a sectional view of a disposable nozzle according to the prior art.

FIG. 2 depicts a schematic representation in a sectional view of a disposable nozzle according to the prior art, mounted in a hand piece.

FIG. 3 depicts a schematic representation in a sectional view of a presently preferred embodiment of a disposable nozzle according to the present invention.

FIG. 4 depicts a schematic representation in a sectional view of a presently preferred embodiment of a disposable nozzle according to the present invention mounted in a hand piece.

FIG. 5 depicts a hand piece according to the present invention with disposable nozzle connected to a control device.

### DETAILED DESCRIPTION OF PRESENTLY PREFERRED EMBODIMENTS

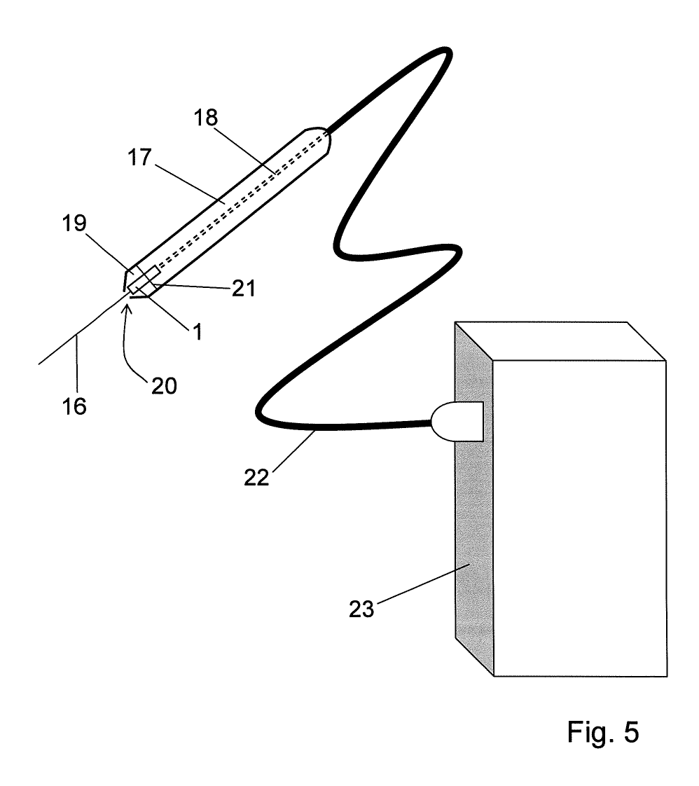

FIG. 5 shows a hand piece 17 according to the present invention which is connected to a control device 23 by means of a high pressure lead 22. From said control device 23, cleaning liquid, in particular brine, is pumped by means of a liquid pump from a tank into high pressure lead 22 and finally into the hand piece 17. Each of hand piece 17 and a hand piece cap 19 defines a cavity therein configured to receive therein a disposable nozzle 1 and may be detachably screwed to each other and opened again by means of a thread 21 to insert the disposable nozzle 1 and to clean the components. In said hand piece 17, the liquid flows to the disposable nozzle 1 in a hand piece duct 18. After the exit from this disposable nozzle 1, the water jet instantaneously enters into the environment. In order to ensure this, the hand piece 17, in particular the hand piece cap 19 arranged at the tip thereof, has to exhibit an aperture region 20 which is short and as wide as possible, so that the high-pressure micro water jet 16 is not diverted by adhesion forces.

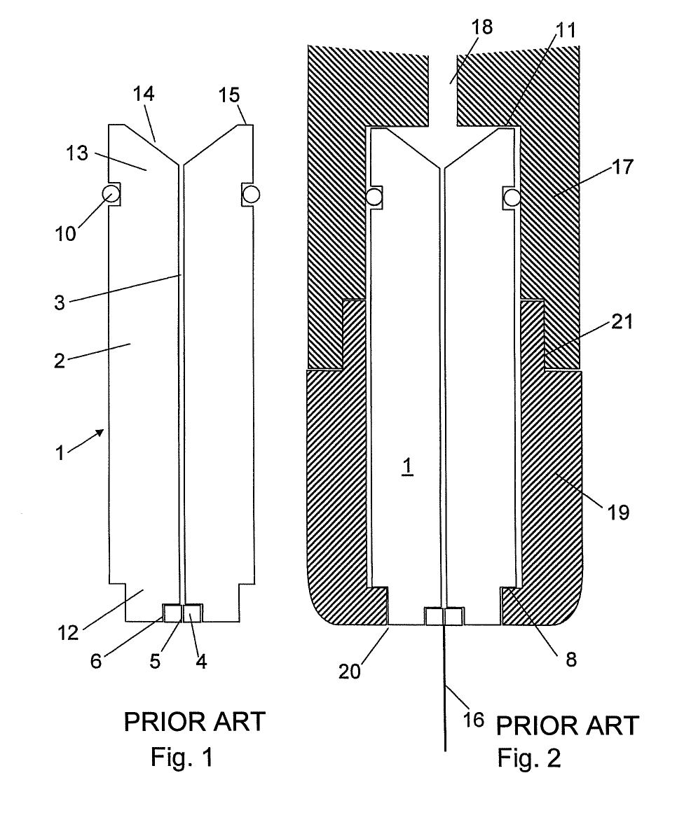

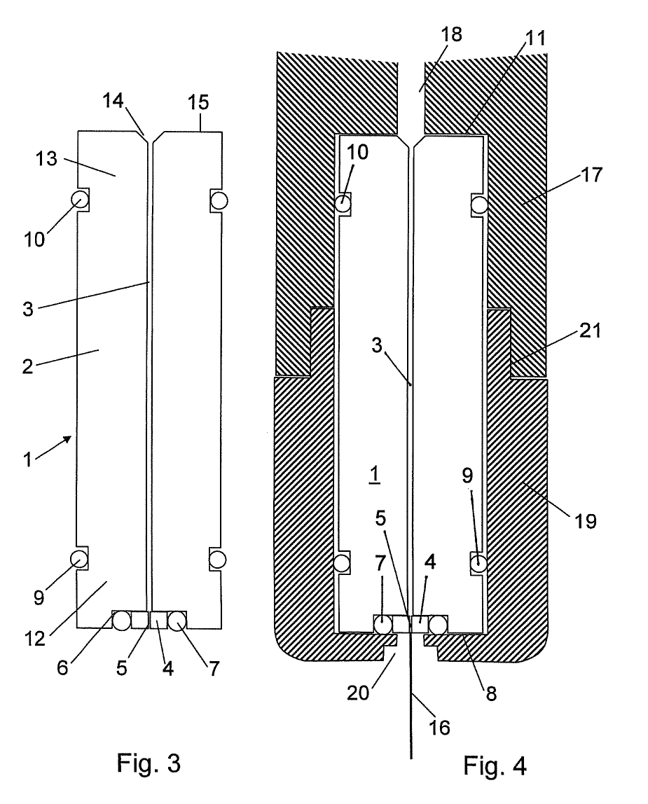

The reference numerals of FIGS. 3 and 4 correspond to those of FIGS. 1 and 2. However, FIG. 3 shows an embodiment of a nozzle body 2 according to the present invention. Nozzle body 2 in FIG. 3 includes a laterally closed nozzle body duct 3 and an orifice 4 having an orifice duct 5 in the front terminal region 12 proximal to the exit of the water jet of the nozzle body 2.

In this embodiment of the present invention according to FIGS. 3 and 4, the orifice 4 is attached in the front side central recess 6 in the nozzle body 2 with an O-ring 7 in a clamping manner. Said O-ring 7 holds the orifice 4 in the nozzle body 2 in the center. Since the disposable nozzle 1 is built into a hand piece and detached only once, the attachment of orifice 4 with the O-ring 7 is sufficient.

On the one hand, the advantage of said O-ring 7 is that now no adhesive is required any longer between orifice 4 and nozzle body 2. Thus, the present invention avoids any uneven distribution of adhesive that might cause a sloped position of orifice 4 in nozzle body 2. Such sloped position of orifice 4 in nozzle body 2 has been known to prevent the highly precise centering of the orifice 4 in nozzle body 2 that is essential for a high quality of micro water jet 16. In the embodiment of the present invention depicted in FIG. 3, orifice 4 is now directly supported in the correspondingly wider front recess 6 in the nozzle body 2. Further, the O-ring 7 is self-centering due to its uniform characteristics, such as thickness, elasticity and compressive modulus along its entire circumference.

In this embodiment depicted in FIG. 4, the nozzle body 2 is not attached at the hand piece cap 19 in a shoulder-sealing manner as is illustrated in FIG. 2. Instead, in the embodiment depicted in FIG. 4, the nozzle body 2 is front sealing in the hand piece cap 19. Correspondingly, the hand piece cap 19 in the embodiment depicted in FIG. 4 has a somewhat small aperture region 20 in the front region. This small aperture region 20 is advantageous in that it is now ensured that orifice 4 cannot be ejected from the nozzle body 2 into the wound, since the orifice 4 is secured now by the front region of hand piece cap 19. In the embodiment of the present invention, the front region of hand piece cap 19 retains orifice 4 and provides a good front sealing by the high pressure in nozzle body duct 3.

In the embodiment depicted in FIG. 4, the O-ring 7 also seals between nozzle body 2 and orifice 4. This O-ring 7 sealing between nozzle body 2 and orifice 4 prevents the liquid from accidentally exiting the nozzle body duct 3 outside orifice 4. The sealing applied by O-ring 7 that prevents liquid from laterally exiting outside of orifice 4, prevents any laterally exiting liquid from exerting an adhesion action onto the micro water jet 16 that might cause an inoperative stream of micro water jet 16. Moreover, in this FIG. 4 embodiment according to the present invention, the aperture region 20 of hand piece cap 19 should be as large and short as possible so as to not perturb micro water jet 16.

In accordance with the present invention, it is desirable that O-ring 7 should slightly protrude from the nozzle body 2. During the assembly into a hand piece 17, the O-ring 7 thereby additionally has a sealing action against hand piece cap 19. This has the advantage that the invasion of bacteria is already prevented in the most frontal region of the hand piece cap 19. As shown schematically in FIG. 4, because of the relative arrangement between orifice 4, hand piece cap 19 and nozzle body 2, the tightness at O-ring 7 increases with increasing pressure behind the micro water jet 16.

In addition to the rear O-ring 10, a further embodiment of the present invention of the disposable nozzle 1 comprises a further frontal O-ring 9 at the cylinder barrel area of the nozzle body 2. On the one hand, O-ring 9 seals between the nozzle body 2 and the hand piece cap 19. On the other hand, O-ring 9 also has a centering function. By means of the two O-rings 9, 10, the nozzle body 2 is guided centrally in the hand piece 17 with its hand piece cap 19. Said guidance prevents a sloped position of the nozzle body 2 in hand piece 17. Preferably, each of the two O-rings 9, 10, has a different color at the cylinder barrel area. This color label facilitates the correct handling for the user and prevents an incorrect insertion of the disposable nozzle 1.

Desirably, disposable nozzles 1 in accordance with the present invention are made from plastics and nonetheless may fulfill the required functions as well as those made of metal. Further, the manufacture thereof is cheaper. Since the disposable nozzles 1 have to be disposed after a single usage, the waste of raw material is also lesser with disposable nozzles made from plastics.

In a further embodiment of the present invention, the nozzle body 2 has a conical aperture 14 at its rear terminal region 13. The conical aperture 14 is just as large as the inner aperture of hand piece duct 18. Desirably, at most the conical aperture 14 constitutes half of the entire rear surface 15 of the nozzle body. Thus, said conical aperture 14 is essentially smaller than the one according to the prior art. On the one hand, this has the advantage that in the smaller conical aperture 14 in accordance with the present invention, less turbulences can build up. On the other hand, the rear surface 15 of the nozzle body 2 in accordance with the present invention thereby becomes larger than was the case in the prior art. The relatively larger rear surface 15 of the nozzle body 2 in accordance with the present invention results in a supporting area to hand piece 17 which is more stable in the assembled state. This facilitates assuring the central position of the disposable nozzle 1 in hand piece 17. Furthermore, it is advantageous if the rear surface 15 is formed in an essentially even manner, in order to rest on a rear stopper 11 of the hand piece 17.

Additionally, the described disposable nozzle 1 according to the present invention has a superior protection against splattering contaminations since the hand piece 19 has a smaller aperture region 20 in the front region. Starting from the exit of the orifice 4, the total angle in the aperture region 20 of the hand piece cap 19 according to the present invention is at least 45°, preferably at least 60°.

The disposable nozzle 1 according to the present invention has been shown to be much safer in comparison to one according to the prior art with respect to the quality of the micro water jet 16. Furthermore, manufacture of the disposable nozzle 1 according to the present invention is cheaper in comparison to one according to the prior art.

### LIST OF REFERENCE NUMERALS

1. Disposable nozzle
2. Nozzle body
3. Nozzle body duct
4. Orifice
5. Orifice duct
6. Recess
7. O-Ring
8. Front stopper
9. Front O-Ring
10. Rear O-Ring
11. Rear stopper
12. Front terminal region of the nozzle body
13. Rear terminal region of the nozzle body
14. Conical aperture
15. Rear surface of the nozzle body
16. Micro water jet
17. Hand piece
18. Hand piece duct
19. Hand piece cap
20. Aperture region of the hand piece cap
21. Thread
22. High pressure lead
23. Control device

1. A disposable nozzle for inserting into a hand piece for treating and cleaning wounds by means of a highly focused high-pressure micro water jet comprising:
a cylindrical nozzle body having a front terminal region proximal to the exit of the water jet, the cylindrical nozzle body having a rear terminal region distal to the exit of the water jet, the cylindrical nozzle body having a centrally traversing nozzle body duct that is laterally closed, the cylindrical nozzle body having a recess which is centrally arranged in the front terminal region of the nozzle body at a front side thereof;
a cylindrical orifice having a centrally traversing orifice duct, wherein the orifice is arranged in such a manner that both ducts adjoin to each other in a coaxial manner; and
an O-ring that is disposed in the recess in a clamping manner between the nozzle body and the orifice, said O-ring retaining the orifice in the center of the nozzle body.

2. The disposable nozzle according to claim 1, wherein said O-ring protrudes from the nozzle body for sealing against a front stopper in the assembled state.

3. The disposable nozzle according to claim 1, wherein the nozzle body has a cylinder barrel area with a front terminal region and a rear terminal region disposed opposite the front terminal region, the disposable nozzle further comprising:
a second O-ring disposed in the front terminal region of the cylinder barrel area of the nozzle body; and
a third O-ring disposed in the rear terminal region of the cylinder barrel area of the nozzle body.

4. The disposable nozzle according to claim 3, wherein each of the second and third O-rings has a different color at the cylinder barrel area.

5. The disposable nozzle according to claim 2, wherein the nozzle body has a cylinder barrel area with a front terminal region and a rear terminal region disposed opposite the front terminal region, the disposable nozzle further comprising:
a second O-ring disposed in the front terminal region of the cylinder barrel area of the nozzle body; and
a third O-ring disposed in the rear terminal region of the cylinder barrel area of the nozzle body.

6. The disposable nozzle according to claim 5, wherein each of the second and third O-rings has a different color at the cylinder barrel area.

7. The disposable nozzle according to claim 1, wherein the nozzle body is made from plastics.

8. The disposable nozzle according claim 1, wherein the nozzle body has a rear surface disposed opposite to the orifice, wherein the nozzle body duct has a rear terminal region that defines a conical aperture, said conical aperture being at most half as large as the entire rear surface of the nozzle body.

9. The disposable nozzle according to claim 1, wherein the nozzle body has a rear surface disposed opposite to the orifice, wherein the rear surface of the nozzle body is configured in an essentially even manner for abutting against a rear stopper in the assembled state.

10. An apparatus for treating and cleaning wounds by means of a highly focused high-pressure micro water jet, the apparatus comprising:
a hand piece defining a cavity therein and having a forward end and a rear end;
a hand piece cap defining a cavity therein, said hand piece cap being detachably connected to said forward end of said hand piece;
a high-pressure lead having one end connected to said rear end of said hand piece;
a disposable nozzle disposed partly in said cavity of said hand piece and partly in said cavity of said hand piece cap, said disposable nozzle including:
a cylindrical nozzle body having a front terminal region proximal to the exit of the water jet, the cylindrical nozzle body having a rear terminal region distal to the exit of the water jet, the cylindrical nozzle body having a centrally traversing nozzle body duct that is laterally closed, the cylindrical nozzle body having a recess which is centrally arranged in the front terminal region of the nozzle body at a front side thereof;
a cylindrical orifice having a centrally traversing orifice duct, wherein the orifice is arranged in such a manner that both ducts adjoin to each other in a coaxial manner; and
an O-ring that is disposed in the recess in a clamping manner between the nozzle body and the orifice, said O-ring retaining the orifice in the center of the nozzle body.

11. The apparatus according to claim 10, wherein the hand piece cap defines an aperture region having a central axis aligned with the central axis of the centrally traversing orifice duct of the cylindrical orifice and wherein starting from the exit out of the cylindrical orifice the total angle in the aperture region of the hand piece cap is at least 45°.

12. The apparatus according to claim 10, wherein the disposable nozzle is assembled within the hand piece and the hand piece cap in a front-sealing manner.

13. The apparatus according to claim 12, wherein the hand piece cap defines an aperture region having a central axis aligned with the central axis of the centrally traversing orifice duct of the cylindrical orifice and wherein starting from the exit out of the cylindrical orifice the total angle in the aperture region of the hand piece cap is at least 45°.

14. The apparatus according to claim 13, wherein starting from the exit out of the cylindrical orifice the total angle in the aperture region of the hand piece cap is at least 60°.

15. The apparatus according to claim 10, wherein said O-ring protrudes from the nozzle body for sealing against the hand piece cap.

16. The apparatus according to claim 10, wherein the nozzle body has a cylinder barrel area with a front terminal region and a rear terminal region disposed opposite the front terminal region, the disposable nozzle further comprising:
a second O-ring disposed in the front terminal region of the cylinder barrel area of the nozzle body; and
a third O-ring disposed in the rear terminal region of the cylinder barrel area of the nozzle body.

17. The apparatus according to claim 16, wherein the second O-ring seals against the hand piece and the third O-ring seals against the hand piece cap.

18. The apparatus according to claim 16, wherein each of the second and third O-rings has a different color at the cylinder barrel area.

19. The apparatus according to claim 10, wherein the nozzle body is made from plastics.

20. The apparatus according claim 10, wherein the nozzle body has a rear surface disposed opposite to the cylindrical orifice, wherein the nozzle body duct has a rear terminal region that defines a conical aperture, said conical aperture being at most half as large as the entire rear surface of the nozzle body.
